(19) United States
(12) Patent Application Publication
(10) Pub. No.: US 2014/0277570 A1
(43) Pub. Date: Sep. 18, 2014

(54) BONE GROWTH PROMOTION SYSTEMS AND METHODS

(71) Applicant: LANX, INC., Broomfield, CO (US)

(72) Inventors: Keyvan Behnam, Simi Valley, CA (US); Jeffrey Thramann, Niwot, CO (US); Elizabeth Perez, Broomfield, CO (US)

(73) Assignee: LANX, INC., Broomfield, CO (US)

(21) Appl. No.: 13/837,595

(22) Filed: Mar. 15, 2013

(51) Int. Cl. A61L 27/36; A61K 47/46; A61L 27/54; A61K 35/32

(52) U.S. Cl. CPC A61L 27/3608; A61K 35/32; A61K 47/46; A61L 27/54
USPC 623/23.51; 424/549; 424/423; 514/7.6; 623/23.63; 156/146; 112/475.08

(57) ABSTRACT

This disclosure describes systems and methods for providing a novel delivery system for implantation in a patient. Further, this disclosure describes a delivery system including a carrier that is made of at least one monolithic bone tissue for retaining one or more substances.

### TECHNICAL FIELD

A delivery system for delivering a substance and/or a material to a patient after implantation is provided. In some embodiments, the substance or material operates to promote bone growth in the patient.

### INTRODUCTION

Bone fractures and orthopedic injuries have a long healing time, during which the fractures or injuries must be immobilized or supported to allow recovery. While bone wounds can regenerate without the formation of scar tissue, fractures and other orthopedic injuries take a long time to heal, during which time the bone is unable to support physiologic loading unaided. The use of bone grafts and bone substitute materials to support injured bone or otherwise promote bone growth in orthopedic medicine is known. For example, metal pins, screws, rods, plates and meshes are frequently utilized to support injured bone. However, metal is significantly more stiff than bone and the use of metal may result in decreased bone density around the implant site due to stress shielding. Bone grafts range from intact bone to particulated bone placed in a carrier. Grafts made of intact bone are inflexible, while grafts made of particulated bone placed in a carrier are flexible. However, flexible bone grafts, typically, require the use of an exogenous carrier, such as a mesh bag. Further, bone grafts are often difficult to handle and may migrate away from the surgical site.

The present disclosure is directed toward overcoming one or more of the problems discussed above.

### BRIEF DESCRIPTION OF THE DRAWINGS

The following drawing figures, which form a part of this application, are illustrative of embodiments of systems and methods described below and are not meant to limit the scope of the disclosure.

FIG. 1 illustrates an embodiment of a cross-sectional view of a delivery system.

FIG. 2 illustrates an embodiment of a cross-sectional view of a delivery system.

FIG. 3 illustrates an embodiment of a cross-sectional view of a delivery system.

FIG. 4. illustrates an embodiment of a cross-sectional view of a pocket filled with one or more substances.

FIG. 5 illustrates an embodiment of a cross-sectional view of a pocket filled with one or more substances.

FIG. 6 illustrates an embodiment of a top view of a delivery system.

FIG. 7 illustrates an embodiment of a side view of a delivery system.

FIG. 8 illustrates an embodiment of a side view of a delivery system.

FIG. 9 illustrates an embodiment of a top view of a delivery system.

FIG. 10 illustrates an embodiment of an isometric view of a delivery system.

FIG. 11 illustrates an embodiment of an isometric view of a delivery system.

FIG. 12 illustrates an embodiment of a side view of a delivery system implanted around a patient's bones.

FIG. 13 illustrates an embodiment of an isometric view of a delivery system.

FIG. 14 illustrates an embodiment of an isometric view of a delivery system.

FIG. 15 illustrates an embodiment of an isometric view of a delivery system.

FIG. 16 illustrates an embodiment of an exploded, isometric view of a pocket being filled with a substance.

FIG. 17 illustrates an embodiment of an exploded, isometric view of a pocket being filled with a substance.

FIG. 18 illustrates an embodiment of an exploded, partial, side view of a delivery system being implanted into a patient's spine.

FIG. 19 illustrates an embodiment of an exploded, partial, side view of a delivery system being implanted into a patient's spine.

FIG. 20 illustrates an embodiment of a partial, side view of a delivery system implanted into a patient's spine.

FIG. 21 illustrates an embodiment of an exploded, partial, side view of a delivery system being implanted into a patient's bone.

FIG. 22 illustrates an embodiment of a partial, isometric view of a delivery system implanted into a patient's bone.

FIG. 23 illustrates an embodiment of a method for treating a bone fracture or defect.

FIG. 24 illustrates an embodiment of a method for forming a delivery system.

FIG. 25 illustrates an embodiment of a kit for forming a delivery system.

FIG. 26 illustrates an embodiment of an exploded, side view of a delivery system.

FIG. 27 illustrates an embodiment of a side view of a delivery system.

FIG. 28 illustrates an embodiment of a method for forming a delivery system.

FIG. 29 illustrates an embodiment of a method for forming a delivery system.

### SUMMARY

This disclosure describes systems and methods for providing a novel delivery system for implantation in a patient. Further, this disclosure describes a delivery system including a carrier that is made of at least one monolithic bone tissue for retaining one or more substances.

### DETAILED DESCRIPTION

Demineralized, as used herein, refers to any material made by removing mineral material from tissue, such as bone tissue, and encompasses all materials described as substantially, partially, or fully demineralized. In some embodiments, the demineralized compositions identified herein contain less than 5% calcium by weight. In other embodiments, the demineralized compositions identified herein contain less than 1% calcium by weight. In further embodiments, the demineralized compositions contain greater than 5% calcium by weight, but contain less calcium than before demineralization.

Bone, as used herein, refers to bone of autogenous, allogeneic, xenogeneic, or transgenic origin, and may be cortical, cancellous, or cortico-cancellous.

Monolithic bone, as used herein, refers to a substantially intact piece or fragment of bone, including but not limited to a whole bone, pieces obtained by mechanical processes (i.e. shaving, slicing, or fracturing impact), sheets, and grown or cultured bone tissues.

Carrier (or covering), as used herein, refers to a material capable of at least partially surrounding a substance, and is made of bone tissue. In some embodiments, a monolithic bone tissue is used, which may be a demineralized monolithic bone. In some embodiments, the monolithic bone is cancellous or cortical bone. In some embodiments, the carrier is made from pieces of demineralized cortical bone, including autogenic, allogeneic, xenogeneic, and transgenic bone. In some embodiments, the carrier is free of non-biocompatible materials, and completely resorbable or completely capable of being incorporated into a surgical site. In one embodiment, the carrier is a wrap or sheet. In some embodiments, the wrap or sheet is made of flexible demineralized monolithic bone. For example, the carrier may be OsteoWrap or OsteoSponge both of which are manufactured by Bacterin (Belgrade, Mont., USA). In other embodiments, the carrier is weight bearing.

The carrier is actively released over time, for example by degradation due to mechanical action, immune activity or enzymatic action. Further, the carrier may be configured to passively release one or more substances retained within the carrier. The carrier may also have a substance applied to its surface by any system or method.

Porous, as used herein, refers to a material that allows materials and substances to travel or diffuse through the material between adjacent spaces. The porous section may be made by any suitable system or method, including but not limited to mechanical, chemical, and enzymatic processes. In some embodiments, the porous section is made by mechanical perforation by a device such as a needle. The pore size and shape may be selected from a plurality of sizes and shapes that operate to give optimal porosity with respect to one or more substances. Optimal porosity is dictated by a plurality of factors including but not limited to the specific injury type and location, desired diffusion rate, treatment substances, and patient characteristics and medical history.

Flexible, as used herein, refers to the property of being flexible, pliable, or elastic. A flexible monolithic bone tissue substantially conforms to the site of surgical implantation.

Weight bearing, as used herein, refers to the property of being load bearing. A weight bearing carrier is capable of helping to support physiologic loading during at least a portion of the healing process and/or bone regeneration process after implantation in a patient. In some embodiments, a weight bearing carrier is substantially inflexible or rigid.

Bone grafting material, as used herein, refers to a material comprising one or more osteoconductive, osteogenic, or osteoinductive materials, compounds, substances, or properties. In some embodiments, the bone grafting material is biocompatible. In other embodiments, the bone grafting material also comprises one or more inert agents. In some embodiments, the bone grafting material comprises demineralized bone matrix, bone chips, bone fragments, bone powder, bone matrix, mineralized allograft, and ceramic biomaterials. In some embodiments, growth factors and other growth enhancing agents are present. In yet other embodiments, the bone grafting material comprises a structurally supporting material. In still other embodiments, the bone grafting material provides a substrate for native cell infiltration and proliferation.

Physical attachment mechanism, as used herein, refers to any mechanism of connecting, placing, inserting, or cementing any embodiment of the delivery system in or on a surgical site, including but not limited to mechanical or chemical methods. In one embodiment, the physical attachment mechanism is one or more conventional surgical attachment mechanisms, such as a string, band, glue, or cement. In another embodiment, the physical attachment mechanism comprises a force exerted on the delivery system by virtue of its placement. In one embodiment, the friction created by pressure exerted on the delivery system by adjacent tissue is the physical attachment mechanism. In one embodiment, the physical attachment mechanism is at least partially or entirely the force exerted on the delivery system by adjacent vertebra or vertebral disks.

As discussed above, bone fractures and orthopedic injuries have a long healing time, during which the fractures or injuries must be immobilized or supported to allow recovery. Previous methods of support or immobilization include the use of bone grafts, ranging from inflexible intact bone grafts to flexible bone grafts of particulated bone placed in a carrier. However, flexible bone grafts typically require the use of an exogenous carrier, such as a mesh bag or polymer, decreasing biocompatibility. Further, these previously utilized flexible bone grafts often migrate away from the surgical site. Additionally, some materials in these grafts may require removal by additional surgery.

Accordingly, the systems and methods described herein provide a delivery system for delivering a substance after implantation. The delivery system comprises a carrier and a substance to be retained and delivered by the carrier. The carrier is made of one or more sheets of monolithic bone tissue or, alternatively, the carrier is a demineralized cancellous bone block with a cavity having an opening. The carrier may include an allograft derived plug that seals the opening of the demineralized cancellous bone block. The delivery system does not require an exogenous carrier. Further, the delivery system does not require the use of any foreign material (materials that are not naturally created by or are not derived from materials created by the patient or patient's species, such as metals, ceramics, and/or polymers). Thus, the delivery system as described herein promotes biocompatibility. Further, because the carrier is made of bone tissue, the carrier itself is actively released or absorbed into the patient over time. Further, the carrier allows for easy sizing and shaping of the delivery system. Accordingly, a delivery system can be shaped and sized based on the patient and/or surgical site. The absorption of the carrier and/or the specific sizing/shaping capability of the carrier mitigate migration of the delivery system after implantation. In some embodiments, the bone tissue carrier prevents migration of the contained graft material after implantation into the patient because of the resorbtion characteristics of the carrier and/or and the specific sizing/shaping capability of the carrier. In other embodiments, the bone tissue carrier reduces the migration of the particulate graft material after implantation into the patient when compared to previously utilized bone grafts that utilized exogenous carriers that are physically mixed with the graft material.

Additionally, the carrier may be flexible, which allows the delivery system to conform to bony surroundings when placed in the surgical site. Further, the carrier provides for easier handling properties, (such as sizing, shaping, forming, and surgical insertion) when compared to previously utilized exogenous carriers.

FIGS. 1-28 illustrate embodiments of a delivery system 100 or portions of a delivery system 100. The delivery system 100 includes a carrier 102 and a substance 104. The delivery system 100 is used to treat a variety of tissue defects or injuries. In some embodiments, the delivery system 100 is used to treat a bone fracture or defect, or otherwise promote bone growth at a desired anatomical location. For example, the delivery system 100 is used in procedures, such as posterolateral fusion, maxofacial reconstruction, dental reconstruction, craniofacial reconstruction, and ridge augmentation, or configured for correcting or treating vertebral compression fractures, adult or pediatric scoliosis, long bone defects, or osteochondral defects. For example, FIG. 22 illustrates a delivery system 100 implanted in a long bone of the patient 118. In some embodiments, the delivery system 100 is utilized to treat a traumatic bone fracture. In other embodiments, the delivery system 100 treats/corrects defects, such as developmental, chronic, progressive, or degenerative bone defects. The defects may be characterized as missing, malformed, or poor quality bone. In other embodiments, the delivery system 100 treats/corrects a tissue defect or injury resulting from surgical intervention. For example, the delivery system 100 may be utilized to promote bone growth between two vertebrae of a patient. In some cases, system 100 may be used in support of a posterior lumbar fusion, an anterior lumbar interbody fusion (ALIF), a transforaminal lumbar interbody fusion (TLIF), lateral interbody fusions, fusions between adjacent spinous processes, and any number of other fusion procedures. Other suitable applications for the delivery system 100 are discussed herein. The listed applications for the delivery system 100 are merely representative and are not intended to be limiting.

For example, if the delivery system 100 is utilized to treat or promote a posterior lateral fusion, the delivery system 100 may be sized to have a height of about 7 cm to overlap the transverse process and a width of about 2.5 cm. These sizes are merely exemplary and are not meant to be limiting. Any suitable size for the delivery system 100 may be utilized based on the patient and surgical site. In some embodiments, the delivery system has a length of 10 cm, width of 2.5 cm and a height from 0.2 cm to 1.5 cm. In other embodiments, the delivery system 100 may be utilized or designed for insertion in a cage. In embodiment, where the delivery system 100 is designed for insertion in a cage, the delivery system may have a length of 1.2 cm, a width of 1.2 cm or 2.0 cm.

In some embodiments, the delivery system 100 is designed to hold, remain intact, and/or support the bone injury or defect for about 2 years, 1.5 years, 1 year, 9 months, 8 months, 7 months, 6 months, 5 months, 4 months, 3 months, 2 months, 1 month, or 2 weeks. In other embodiments, the delivery system 100 is designed to hold, remain intact, and/or support the bone injury or defect for 1 to 6 months, 2 to 6 months, 3 to 6 months, 4 to 6 months, 5 to 6 months, 1 to 5 months, 1 to 4 months, 1 to 3 months, 1 to 2 months, 2 to 5 months, 2 to 4 months, 2 to 3 months, 3 to 5 months, 3 to 4 months, or 4 to 5 months. In further embodiments, the delivery system 100 is designed to hold, remain intact, and/or support the bone injury or defect for up to 1 month, 2 months, 3 months, 4 months, 5 months, 6 months, 7 months, 8 months, 9 months, 1 year, 1.5 years, or 2 years.

In some embodiments, the carrier 102 is at least one sheet of monolithic bone tissue. In alternative embodiments, the carrier 102 is a demineralized cancellous bone block with a cavity having an opening. An allograft derived plug may be used to seal the opening of the demineralized cancellous bone block. Accordingly, the carrier 102 has useful biological properties that promote healing and result in favorable material release kinetics. In one embodiment, a carrier 102 comprising demineralized bone is configured to degrade and release a substance 104. In some embodiments, the carrier 102 is a bone grafting material.

The carrier 102 is biocompatible and resorbable, and degrades or is absorbed into the body after implantation. Therefore, the carrier 102 actively releases materials into the patient. Further, the carrier 102 may be porous allowing the carrier 102 to passively release one or more substances 104 retained within the carrier 102 to the patient 118. However, the carrier 102 may facilitate active and/or passive release by any suitable system or method, including but not limited to physical and/or chemical processes.

Active phase characteristics comprise the release of bone grafting materials from the carrier surface, such as degradation and adsorption of the monolithic bone carrier 102 into a surgical site. The carrier 102 is a bone grafting material because the carrier 102 is made of monolithic bone. The carrier 102 may also contain osteoconductive, osteogenic, and/or osteoinductive compounds. Further, the carrier 102 may be treated or impregnated with additional bone grafting materials, such as allogeneic demineralized bone graft, mineralized allograft bone chips, autograft bone derived from the patient, metabolically active cells, growth factors and growth enhancing agents that are located on the surface or throughout the carrier 102. The carrier 102 may further include other bone grafting materials, bone morphogenic proteins (BMPs), growth factors, antibiotics, angiogenesis promoting materials, or drugs.

The mechanisms of active release include any chemical, physical, or other force that causes wear or dissolution of the carrier 102. For example, repeated impacts or contact with the bone or other tissue of a surgical site may cause the carrier 102 to degrade. Bodily movement may cause rotational and conformational stresses that result in the degradation of the carrier 102. Additionally, host tissue immunological and inflammatory cells and substances 104 will degrade the carrier 102. Also, the general physiological conditions at the surgical site will result in degradation of the carrier 102.

Due to differential distribution or degradation, substances 104 may be released at different rates at different surface locations. It is possible to predict the wear of different areas of the carrier 102 and distribute substances 104 preferentially to sites that wear more or less, depending on whether it is preferential to have a quick or slow release carrier 102.

For example, the carrier 102 may be resorbable, and degrade after implantation to a surgical site, releasing said substances 104. The carrier 102 may be partially or entirely resorbable, depending on injury prognosis and desired methods of treatment. If the carrier 102 is partially resorbable, carrier 102 may be biocompatible and suitable for long-term implantation. A partially resorbable carrier 102 may be formed of a combination of bone-tissue derived material and any natural or synthetic structure useful to form a carrier 102. The resorbable portion of the carrier 102 may be configured to release a substance 104 over a time period and in an amount that promotes healing. For example, the origin, thickness, material, and other features of the carrier 102 may be varied to reach a desired degradation or resorption life, depending on the injury and patient characteristics.

The carrier 102 is configured to release one or more substances 104. In some embodiments, the carrier 102 has a passive release mechanism that facilitate the transfer or diffusion of materials into and out of the carrier 102, such as holes, pores, mesh, or gaps that allow substances 104 to exit the delivery system 100. The passive mechanism may be size discriminatory, allowing timed or preferential release of one or more substances 104 based on molecule size or aggregated size. Substances 104 may be released in amounts that tend to increase a physiological healing response, taking into consideration the type of injury and elapsed time since injury. The release sites of the carrier 102 may be continuous over the entire the carrier 102, or may be present in one or more parts or sections of the carrier 102. The carrier 102 may comprise multiple passive release mechanisms and sites, permitting any combination of passive delivery mechanisms. In some embodiments, different release sites have different discriminatory release mechanisms, for example differently sized spacing or pores, which allow substance release characteristics to vary over the surface of the delivery system 100. The configuration of the different release mechanisms will vary with the physical and chemical properties of the substances 104, and may take medically relevant factors into consideration. The configuration may be selected based on desired release characteristics, and may be adjusted by varying the basic structure, shape, thickness, permeability, porosity, strength, flexibility, elasticity, or other features. Some of these features or changes to features may be interdependent or covariant. Such multiple discriminatory release mechanisms may be configured to promote the healing of tissues that vary in type, strength, injury severity, or healing time.

In some embodiments, the passive mechanism allows cellular infiltration and tissue growth within the carrier 102. In some embodiments, one or more selectively permeable portions comprise pores of a size large enough to permit compounds and small molecules to cross the carrier 102, but small enough to prevent cellular infiltration. In some embodiments, the carrier 102 facilitates cell growth through the carrier 102, such as through gaps, pores, or spaces. In these instances, the carrier 102 may be size selective, allowing cellular infiltration by specific host cells. For example, the pore size may be approximately 0.45 microns, a size that allows selective small molecule diffusion. In some embodiments, the size of the pores is from 100 microns to 500 microns. These pore sizes prevent graft materials from migrating away.

For example, the thickness of the carrier 102 may be changed over any part of the carrier 102 surface to achieve desired release kinetics. The release rate and kinetics of carrier 102 and/or substance 104 may be varied depending on medically relevant criteria. In some embodiments, for example, carrier 102 and/or substances 104 may be released immediately and entirely upon implantation. In other embodiments, carrier 102 and/or substances 104 may be released evenly and continuously throughout the degradation process. In yet other embodiments, carrier 102 and/or substances 104 are released in a manner on the continuum having the previous embodiments as end points. In some embodiments, the carrier 102 and/or substances 104 may be released at different rates at different surface locations. It is also contemplated that the carrier 102 and/or substance release rates may be selected to vary over time, and/or may naturally vary over time. Also contemplated are variations in the carrier 102 and/or substance 104 identity or combinations over the surface and over time.

In some embodiments, the at least one sheet of monolithic bone tissue is made of demineralized bone particles. The demineralized bone particles may be cortical bone or cancellous bone. The cancellous bone is more porous than the cortical bone. In some embodiments, the carrier 102 is mechanically perforated to increase the porosity of the carrier 102. The perforations 114 may be sized and/or spaced to control the diffusion rate of one or more substances 104 or of cells across the carrier 102. For example, the carrier 102 may be mechanically perforate utilizing a drill, hole punch, laser, laser drill, needles, and/or a block of needles. In some embodiments, the perforations 114 or pores 114 are around 100 microns. Patient cells are often around 10 microns; therefore, pores 114 of around 100 microns allow patient cells to pass across the carrier 102 through the pores 114 and contact the retained one or more substances 104. Further, after the carrier 102 is actively released to the patient 118 or fully absorbed or degraded by the patient 118, any substances 104 that were previously retained within the carrier 102 and that were not already passively released to the patient are actively released to the patient 118. In other embodiments, the perforations 114 or pores 114 are from 10 microns to 1 mm. In some embodiments, the perforations 114 or pores 114 are 10 microns, 500 microns, or 1 mm.

In various embodiments, the carrier 102 may have one or more structural characteristics suitable for a specific surgical application, such as being rigid, flexible, elastic, or non-elastic. The carrier 102 may or may not be load bearing, depending on the specific application. In some embodiments, the carrier 102 may change shape or expand after implantation, for example by restoring a shape, size, or configuration after compression or exposure to moisture, temperature, or other bodily conditions or substances 104. Either the carrier 102 or the substances 104 retained by the carrier 102 may incorporate such features and materials.

In some embodiments, the carrier 102 is flexible. The flexibility or rigidity of the carrier 102 that is a sheet of monolithic bone tissue is determined by controlling the thickness of the sheet. The thicker the sheet, the less flexible the carrier 102. Accordingly, the thinner the sheet, the more flexible the carrier 102. Further, the flexibility or rigidity of the carrier 102 may be controlled by demineralizing the carrier 102. The more demineralized the carrier 102, the more flexible the carrier 102. The less demineralized the carrier 102, the less flexible the carrier 102. Accordingly, the flexibility of the carrier 102 can be optimized by controlling the thickness of the sheet of bone tissue and/or by demineralizing the monolithic bone. For example, the carrier 102 may need to be weight bearing. Accordingly, the sheet thickness can be increased and/or the monolithic bone of the carrier may not be demineralized to achieve the desired amount of weight bearing necessary to support an injured or deformed bone to heal after implantation in the patient. A weight bearing carrier 102 is sized and shaped prior to implantation.

For example, cancellous bone is flexible if the thicknesses of the cancellous bone is below 10 mm. Further, cancellous bone is only minimally weight bearing after demineralization. In some embodiments, the cancellous bone is utilized at a thickness of 5 mm. In an additional example, cortical demineralized bone matrix is flexible when the cortical demineralize bone matrix has a thickness of less than 3 mm. Further, cortical demineralized bone matrix provides some weight bearing properties when the cortical demineralized bone matrix has a thickness of 5 mm or more. Mineralized cortical bone is weight bearing at just about any thickness.

The carrier 102 is easily handled. For example, the carrier 102 may be cut with scissors or other cutting tools for easy size and shaping. Further, a flexible carrier 102 is easily bent. The easy sizing and shaping of the carrier 102 allows the carrier 102 to be specifically sized and/or shaped based on the patient and/or surgical site. In some embodiments, the sizing/shaping property of the carrier 102 allows the carrier 102 to prevent migration of the delivery system 100. In other embodiments, the sizing/shaping property of the carrier 102 allows the carrier 102 to reduce migration of the delivery system 100 when compared to previously utilized delivery systems 100 that have exogenous carriers 102.

In some embodiments, one sheet of monolithic bone tissue is folded upon itself and sealed to create a pocket 110. The pocket 110 has an opening 112 or slit 112 that provides access to the interior of the pocket 110. The remaining sides of the pocket 110 are sealed. The one or more substances 104 are inserted into the pocket 110 via the opening 112. Once the one or more substances 104 are inserted into the pocket 110, the opening 112 of the pocket 110 is sealed to create a carrier 102 retaining the one or more substances 104 within a compartment 108.

In some embodiments, the carrier 102 is made from multiple pieces of monolithic bone forming a larger structure, for example by weaving, braiding, adhesion, layering, fusing, binding, or stapling. For example, a plurality of sheets of monolithic bone tissue may be sealed to create a pocket 110. The plurality of sheets may vary in flexibility or all retain substantially the same flexibility. Again once the pocket 110 is filled with the one or more substances 104, the opening 112 is sealed to create a carrier 102 retaining the one or more substances 104 within a compartment 108. For example, a first flexible sheet 102a may be sealed to a second flexible sheet 102b create a pocket 110 as illustrated in FIGS. 1 and 3.

In an alternative embodiment, one or more sheets are sealed to create a compartment 108. For example, a first flexible sheet may be sealed to a second flexible sheet to create a compartment 108. In another example, a first flexible sheet is folded upon itself and sealed to create a compartment 108. Once sealed a slit or opening 112 is cut into the carrier 102 to form a pocket 110.

In another embodiment, the carrier 102 is a demineralized cancellous bone block 122 with a cavity 111 having an opening 112, and an allograft derived plug 120 that seals the opening 112 of the demineralized cancellous bone block 122 as illustrated in FIGS. 26-28. To form this carrier 102, cancellous bone block is demineralized until the desired flexibility is reached. In some embodiments, the demineralized bone block 109 has a similar flexibility to a sponge. The demineralize bone block may be any desirable size and shape. In some embodiments the demineralized bone block 109 is sized and shaped based on the implantation site. In some embodiments the demineralized bone block 109 is rectangular or square.

Next, a cavity 111 is formed in the demineralized bone block 109 to form a demineralized cancellous bone block 122 with a cavity 111 that has an opening 112. The cavity 111 may be manually or automatically created by a specialized cutting tool. For example, in some embodiments, the cutting tool utilized to form the cavity 111 is a cylindrical rotating blade.

Once the cavity 111 is formed, one or more substances 104 are placed in the cavity 111 through the opening 112. The one or more substances 104 as discussed above and below may be numerous different materials. However, in some embodiments, the one or more substances 104 are autograft tissue or cells, demineralized cortical bone powder or fibers, and/or cells derived from allograft tissue. In some embodiments, the cells are periosteal cells, osteoblasts, mesenchymal stem cells, osteoblasts, mesenchymal stem cells, and/or cells that are derived from the amnion or chorionic layer of the placenta. In other embodiments, the one or more substances 104 added to the cavity 111 include cortical demineralized bone powder, mineralized bone chips, synthetic calcium phosphates, glycosaminoglycans or any other substances that increase the volume specific density, compression resistance, and/or the hydration capacity of the demineralized cancellous bone block 122.

Once the substance 104 is stored within the cavity 111 of the demineralized cancellous bone block 122, an allograft derived plug 120 is inserted into the opening 112 to seal the cavity 111 to create a container 108 in a delivery system 100. The allograft derived plug 120 is derived from a demineralized or mineralized bone or from cartilage. The allograft plug 120 is derived from donor tissue or from tissue taken from the patient. The plug 120 is shaped or designed to conform to the opening 112 of the cavity 111 in order to seal or close the cavity 111. The plug 120 prevents substances 104 placed inside the cavity 111 from escaping from the cavity 111 once the plug 120 is inserted in the opening 112. In some embodiments, the plug is derived from the tissue removed from the bone block 109 to create the cavity 111. The formed delivery system 100 is then implanted at the desired surgical site of the patient.

In alternative embodiments, the plug 120 is inserted into the opening 112 of the cavity 111 of the demineralized cancellous bone block 122 without placing any substances 104 in the cavity 111. In this embodiment, the one or more substances 104 are inserted into the cavity 112 after the cavity 112 is sealed with allograft derived plug 120. In some embodiments, the one or more substances 104 are inserted into the cavity 112 through a hollow needle that is inserted into the cavity 112 via the demineralized cancellous bone block 122 or the allograft derived plug 120. The hollow needle injects the one or more substances into the cavity 112 to form the delivery system 100. The hollow needle may form a perforation in the demineralized cancellous bone block 122 or the allograft derived plug 120. Again, the formed delivery system 100 is then implanted at the desired surgical site of the patient.

A delivery system 100 comprising a carrier 102 including a demineralized cancellous bone block 122 with a cavity 111 having an opening 112, an allograft derived plug 120 that seals the opening 112, and a substance 104 of demineralized cortical bone power or fibers absorbs more liquid, such as bone morrow aspirate, blood, growth factor, cell suspension, and/or etc., has an increased matrix density and improved hydration capacity than other delivery systems that utilize a cancellous bone block of the same dimensions. Further, a delivery system 100 comprising a carrier 102 including a demineralized cancellous bone block 122 with a cavity 111 having an opening 112, an allograft derived plug 120 that seals the opening 112 and substance 104 of demineralized cortical bone power or fibers has a higher osteocinductive capacity when compared to other delivery systems that utilize a cancellous bone block of the same dimensions. Further, the delivery system 100 with a carrier 102 including a demineralized cancellous bone block 122 with a cavity 111 having an opening 112, and an allograft derived plug 120 that seals the opening 112 prevents cells from migrating away from the implantation site.

The sealing mechanism 106 may be any suitable system or method for sealing bone tissue, such as an adhesion seal 106a, pressure seal 106b, sutures 106c, staples, a heat seal 106d, a volatile compound seal, and/or various combinations thereof. In some embodiments, the seal mechanism 106 is an adhesion seal 106a and/or heat seal 106d to prevent introducing any foreign or exogenous materials into the patient 118. For example, a carrier 102 may be sealed by applying pressure and heat. The carrier 102 may be held together by overlap, adhesion, self-adhesion, or mechanical mechanisms such as a string, tie, or staple. In some embodiments, a shape is formed by wrapping the carrier 102 around itself and forming a pocket 110 for one or more substances 104. As discussed above, the sealing mechanisms 106 do not require the use of exogenous adhesives or chemical processes to maintain the shape.

The carrier 102 may comprise one or more configurations selected from a plurality of configurations suitable for implantation. In some embodiments, the carrier 102 is configured to heal a bone fracture or defect. Because the one or more sheets of monolithic bone tissue, this type of a carrier is easily cut, sized, and shaped, the carrier 102 can be easily designed to any desired size or shape for placement in the patient at various different surgical sites. Further, a flexible carrier 102 will conform to the surrounding bony contours once implanted in the patient. For example, a flexible carrier 102 allows the delivery system 100 to fit in various surgical sites. FIGS. 12, 19, and 20 illustrate various embodiments of a delivery system 100 conforming to bony parts or portions of a patient 118. In some embodiments, the carrier 102 is pillow shaped, ring shaped, cylinder shaped, rectangular shaped, sphere shaped, square shaped, tube shaped, triangular shaped, funnel shaped, or any other suitable shape for insertion at a surgical site of patient. For instance, FIGS. 6-11, 13-15, 21, and 22 illustrate embodiments of various different shapes of the delivery system 100. In some embodiments, the delivery system 100 is rectangular in shape. In embodiments where the delivery system is rectangular in shape, the delivery system has a size of 12 mm×12 mm×20 mm. In further embodiments, a rectangular delivery system that has a size of 12 mm×12 mm×20 mm may be utilized in interbody fusion. In other embodiments, the where the delivery system is rectangular in shape, the delivery system has a size of 100 mm×25 mm×10 mm. In further embodiments, a rectangular delivery system that has a size of 100 mm×25 mm×10 mm may be utilized in a posterior lumbar fusion.

The carrier 102 may be configured to form single or multiple compartments 108, or may be configured as a sheet or other open shape capable of at least partially retaining a substance 104. The size, weight, flexibility, structural support, and number of compartments 108 may vary to suit a specific injury or specific substance 104 or combination of substances 104. In some embodiments, characteristics of the carrier 102 may be varied within the same compartment 108 and/or across multiple compartments 108. Compartments 108 may share a carrier wall, or may be separated along a string, band, mesh, or other device.

The one or more substances 104 may be any suitable chemical composition or combinations of chemical compositions. For example, the substance 104 may have biological activity or have no substantial biological activity. A substance 104 having biological activity may include any compound or entity that alters or otherwise affects biological or chemical events. Further, for example, the substance 104 may be drugs, coverings, fillers, organic material, inorganic material, biologically derived materials, metals, air or other gasses, and structural materials. In some embodiments, the substance 104 is a bone grafting material. In some embodiments, the substance 104 is osteogenic, osteoinductive, or osteoconductive. In further embodiments, the substance 104 comprises demineralized bone matrix. In other embodiments, the substance 104 is a known pharmaceutical composition. In additional embodiments, the substance 104 comprises one or more growth factors, cytokines, or extracellular matrix molecules. In some embodiments, the substance 104 is demineralized cortical bone powder or fibers. In further embodiments, the one or more substance 104 is cortical demineralized bone powder, mineralized bone chips, synthetic calcium phosphates, and/or glycosaminoglycans.

In additional embodiments, the one or more substances 104 are autograft or allograft cells. In some embodiments, autograft and/or allograft cells are derived from the patient or from a donor. For example autograft and/or allograft cells may be derived from a patient's bone marrow in the form of bone marrow aspirate. In other embodiments, autograft and/or allograft cells are derived from fat tissue, blood in the form of platelet rich plasma, or perisosteum. In some cases these cells are harvested and directly placed in the delivery system 102. In other cases, the cells will be expanded and selected in culture. In some embodiments, the autograft cells are mesenchymal stem cells, preosteoblasts, osteoblasts or chondorblasts. In other embodiments, the autograft and/or allograft cells are in the form of derived mesenchymal stem cells, bone marrow stromal cells, osteoblasts, preosteoblasts, periosteal cells, fat derived cells, and cartilaginous cells. In further embodiments, the autograft and/or allograft cells are trapped within an extracellular matrix. For example, osteocyte cells may be trapped within a bone osteoid, chondrocytes cells may be trapped within cartilage, or periosteal cells or cells derived from amnion or chorion are trapped within an extracellular matrix secreted by the periosteal cells or cells derived from amnion or chorion. Combinations of substances 104 may be chosen to provide desired medical effects based on the specific type of injury, patient characteristics, or any other medically relevant factor or combination of factors.

In some embodiments, the device also comprises a marking agent, such as a radiopaque material, that allows identification of the orientation of the device when implanted. In embodiments, said marking agent is biocompatible and does not require surgical removal.

In some embodiments, the delivery system 100 includes an attachment mechanism 116. The attachment mechanism 116 is used to retain the covering at the surgical site and any mechanisms capable of doing so may be used. The attachment may be to bone or to adjacent tissues such as muscle, tendon, or ligament. Where the covering retains a bone graft substance, the covering may be held in a relatively stable position relative to bone (or relative to the surgical site or surgical defect) to promote bone growth. Accordingly, in some embodiments, the delivery system 100 may be suitable for assisting in attaching tendons, artificial tendons, or ligaments to bone or other structure.

The bone or soft tissue to which the covering is attached may be prepared for receiving the attachment mechanism(s) 116. For example, in spinal applications, slots or perforations may be provided in posterior elements such as transverse processes, spinous processes, or other bone or tissue to receive the attachment mechanism 116.

Any suitable attachment mechanism 116 may be used, including mechanical, physical, chemical, and biological attachment mechanisms 116. The attachment mechanism 116 may be provided at an end of the covering, centrally in or on the covering, generally in or on the body of the covering, or any combinations of these. U.S. Pat. No. 5,899,939 describes attachment mechanisms 116 that may be adapted for use with a covering as provided herein, and is herein incorporated by reference. When an attachment mechanism 116 is used to couple first and second coverings to one another, such attachment or coupling may be done pre-implantation or post-implantation. In some embodiments, the covering may be provided with attachment mechanisms 116 to facilitate suturing or other attachment of the covering in vivo.

In some embodiments, a covering may include an area for receipt of an attachment mechanism 116. For example, a covering may include a tab for receipt of a screw. In other embodiments, an attachment mechanism 116 may interface with any portion of the covering. For example, a screw attachment mechanism 116 may be threaded through a covering at any location, including central to a containment area of the covering. In some embodiments, a screw attachment mechanism 116 may be threaded through the covering and the substance 104 provided in the containment area of the covering.

A further method of attachment may comprise suturing or otherwise attaching the covering to a tether, anchor, or screw embedded in a bony structure, e.g. a pedicle screw of a spinal stabilization system. Such screw, anchor, or tether may pass through the covering and its contained contents to provide fixation, or through a tab at a margin of the covering, or through other structure of the covering.

Further physical attachment mechanisms 116 suitable for use, for example, may comprise wraps, sutures, wires, strings, elastic bands, cables, ropes, chains, plastic wrap, strap, tie, cable tie, or other mechanisms. These mechanisms may be coated, such as with plastic. In some embodiments, a plurality of mechanisms, such as a multiple parallel wires, may be joined together, for example with a plastic strip coating. Suitable techniques for using such attachment mechanisms 116 may include suturing, stitching, knotting, twisting, cinching, knot tying, and similar techniques. Any of these physical fastening materials may be made of any suitable material, including metal, tissue such as allograft, xenograft, autograft, collagen, any other suitable materials or combinations of these.

Chemical attachment mechanisms 116 may comprise, for example, a bioadhesive or glue, cement, tape, tissue adhesives, or similar mechanism. Chemical attachment mechanisms 116 may further comprise mechanisms that facilitate cross-linking. In further embodiments, attachment mechanisms 116 such as crimping, welding, soldering, or brazing may be used. Further, attachment may be achieved via friction. For example, the friction created by pressure exerted on the delivery system by adjacent tissue creates the physical attachment mechanism. In other embodiments, the physical attachment mechanism is the force exerted on the delivery system by adjacent vertebra or vertebral disks.

In some embodiments, biological attachment may be via mechanisms that promote tissue ingrowth such as by a porous coating or a hydroxyapatite-tricalcium phosphate (HA/TCP) coating. Generally, hydroxyapatite bonds by biological effects of new tissue formation. Porous ingrowth surfaces, such as titanium alloy materials in a beaded coating or tantalum porous metal or trabecular metal may be used and facilitate attachment at least by encouraging bone to grow through the porous implant surface. These mechanisms may be referred to as biological attachment mechanisms 116.

Generally, any combination of mechanical, physical, chemical, or biological attachment mechanisms 116 may be used.

Any of the various attachment mechanisms 116 may be provided as part of the covering or may be supplied separately. In various embodiments, the attachment mechanisms 116 may be integral to the covering. Alternatively, the attachment mechanisms 116 may be secured to the covering, for example, by stitching, welding, crimping, or other securing mechanisms. The attachment mechanisms 116 may have any suitable geometric configuration and may optionally include apertures for receiving other components for coupling in vivo, such as an aperture for receiving a screw. Thus, for example, an attachment mechanism 116 may be provided configured for receiving an anchor for fixation to bone. Any number of attachment mechanisms 116 may be provided at any suitable location on the covering.

The attachment mechanism 116 may be manufactured of the same material as the portion of the covering to which attachment mechanism 116 is coupled or may be manufactured of a different material from the portion of the covering to which attachment mechanism 116 is coupled. The attachment mechanism 116 may be resorbable or nonresorbable. The material of the attachment mechanism 116 may be selected to allow anchoring the covering to an adjacent covering having a complementary attachment mechanism 116 or to another structure. In various embodiments, the attachment mechanism 116 may comprise bone grafting materials, allograft, synthetic materials, demineralized bone, nondemineralized bone, other material, or combinations of these materials. The shape and size of the attachment mechanism 116 may be selected based on application.

In some embodiments, the attachment mechanism 116 does not comprise any exogenous material. Accordingly, the delivery system 100 promotes biocompatibility. Further, in some embodiments, the absorption of the carrier 102 and/or attachment mechanism 116 prevents the delivery system 100 from migrating after implantation in the patient. In other embodiments, the absorption of the carrier 102 and/or attachment mechanism 116 reduces migration of the delivery system 100 after implantation in the patient when compared to previously utilized delivery systems 100 that utilized exogenous materials.

FIG. 25 illustrates an embodiment of a kit 500 for treating a bone fracture or defect. The kit 500 includes at least one sheet of monolithic bone tissue 502, one or more substances 104, and a seal mechanism 106. The at least one sheet of monolithic bone tissue 502 is configured to receive the one or more substances 104. The seal mechanism 106 is designed to seal the at least one sheet of monolithic bone tissue 502 to form a compartment 108 retaining the one or more substances 104.

FIG. 23 illustrates an embodiment of a method 200 for treating a bone fracture or defect. As illustrated, method 200 includes a placing operation 202. During the placing operation 202, method 200 places a delivery system comprising a carrier and at least one substance retained in the carrier at the site of a bone fracture or defect. As discussed above, the carrier is at least one sheet of monolithic bone tissue. The least one substance is a bone grafting material.

FIG. 24 illustrates an embodiment of a method 300 for forming a delivery system. FIG. 29 also illustrates an embodiment of a method 400 for forming a delivery system.

As discussed above the delivery system provides numerous advantages, such as being exogenous material free, being foreign material free, promoting biocompatibility, decreasing or preventing migration of the delivery system after implantation, being easily sized and/or shaped, and/or providing easier handling properties, (such as sizing, shaping, forming, and surgical insertion) when compared to previously utilized exogenous carriers. Further as discussed above, the delivery system is used to treat a variety of tissue defects or injuries.

As illustrated, method 300 includes a forming operation 304. During the forming operation 304, method 300 forms a pocket in at least one sheet of monolithic bone tissue. In some embodiments, the monolithic bone is demineralized monolithic bone. In some embodiments, the monolithic bone is demineralized cortical bone, including autogenic, allogeneic, xenogeneic, and transgenic bone. Further, the monolithic bone is free of non-biocompatible materials, and completely resorbable or completely capable of being incorporated into a surgical site. In one embodiment, the monolithic bone is a wrap or sheet. In some embodiments, the wrap or sheet is made of flexible demineralized monolithic bone. For example, the monolithic bone may be OsteoWrap, manufactured by Bacterin (Belgrade, Mont., USA). In other embodiments, the monolithic bone is weight bearing and/or flexible. In some embodiments, the at least one sheet of monolithic bone tissue is made of demineralized bone particles. The demineralized bone particles may be cortical bone or cancellous bone. The monolithic bone is easily handled. For example, the monolithic bone may be cut with scissors for easy size and shaping. Further, a flexible monolithic bone is easily bent.

In some embodiments, the forming operation 304 includes sealing a plurality of sheets of monolithic bone tissue together to form a pocket. For example, the forming operation 304 may include sealing a first sheet of monolithic bone tissue to a second sheet of monolithic bone tissue. In other embodiments, the forming operation includes sealing a first portion of a first sheet of monolithic bone tissue to a second portion of the first sheet of monolithic bone tissue. For example, a flexible sheet of monolithic bone tissue may be folded over on itself and sealed to create pocket. In other embodiments, the forming operation 304 includes sealing a first portion of a sheet of monolithic bone tissue to a second portion of the first sheet of monolithic bone tissue to form a compartment, and slicing a slit into the compartment to form the pocket. In additional embodiments, the forming operation 304 includes sealing a plurality of sheets of monolithic bone together to form one or more compartments, and slicing a slit into the one or more compartments to form one or more pockets. For example, the forming operation 304 may include sealing a first sheet of monolithic bone tissue to a second sheet of monolithic bone tissue to form a compartment, and slicing a slit into the compartment to form the pocket. The pocket has an opening or slit that provides access to the interior of the pocket. The remaining sides of the pocket are sealed. The compartment is space or area within the monolithic bone that is completely enclosed by the monolithic bone.

In some embodiments, method 300 includes a perforation operation 302. During the perforation operation 302, method 300 perforates the monolithic bone to increase the porosity of the bone. The monolithic bone may be perforated prior to or directly after the performance of forming operation 304. The perforations may be sized and/or spaced to control the diffusion rate of one or more substances 104 or of cells across the monolithic bone tissue. For example, the monolithic bone may be mechanically perforated utilizing a drill, hole punch, laser, laser drill, needles, and/or a block of needles. In some embodiments, the perforations or pores are sized around 100 microns.

Next method 300 includes an inserting operation 306. During the inserting operation 306, method 300 inserts at least one substance in the pocket. The at least one substance may be inserted into the pocket by any suitable system or method for implanting a substances into a pocket. For example, the one or more substance may be inserted via an insertion tool. Various insertion tools (also referred to as delivery devices) may be utilized to insert the one or more substances into a pocket. Such insertion tools may include, for example, a delivery gun, a scraper, an injector instrument, pipet, syringe, or other suitable tool. Such tools may be provided separately or may be provided as part of a kit.

Further, method 300 includes a sealing operation 308. During the sealing operation 308, method 300 seals the pocket to form a carrier retaining the at least one substance. Any suitable sealing mechanism may be utilized by method 300 for creating or sealing the pocket, such as the sealing methods described above. In some embodiments, the sealing does not include any exogenous materials and/or any foreign materials. The carrier is similar to the carrier described above.

As illustrated in FIG. 29, method 400 includes a forming operation 404. During the forming operation 404, method 400 forms a cavity in a demineralized cancellous bone block. The formed cavity has an opening. In some embodiments the forming operation 404 is performed by a specialized cutting tool. In some embodiments, the specialized cutting tool is a cylindrical rotating blade.

Next method 400 includes an inserting operation 306. During the inserting operation 406, method 400 inserts at least one substance in the cavity through the opening. The at least one substance may be inserted into the cavity by any suitable system or method for implanting a substances into a pocket. For example, the one or more substance may be inserted via an insertion tool. Various insertion tools (also referred to as delivery devices) may be utilized to insert the one or more substances into the cavity. In some embodiments, the surgeon utilized his gloved hand as the insertion tool. In other embodiments, the insertion tool includes, for example, a delivery gun, a scraper, an injector instrument, pipet, syringe, or other suitable tool. Such tools may be provided separately or may be provided as part of a kit. In some embodiments, the one or more substance is demineralized cortical bone powder or fibers. In some embodiments, the one or more substance is one or more autograft and/or allograft cells.

Further, method 400 includes a sealing operation 408. During the sealing operation 408, method 400 seals the opening of the cavity with an allograft derived plug to form a carrier retaining the at least one substance. Any suitable sealing mechanism may be utilized by method 400 for plugging or sealing the cavity, such as the sealing methods described above. In some embodiments, the sealing does not include any exogenous materials and/or any foreign materials. In some embodiments, the pressure of the inserted plug in the opening seals off the opening. The allograft derived plug is similar to the allograft derived plug described above.

In some embodiments, the carrier is sized and shaped to fit the surgical implantation site. In other embodiments, the carrier is rectangular or square in shape. Further, the carrier is free of non-biocompatible materials, and completely resorbable or completely capable of being incorporated into a surgical site. In other embodiments, the carrier is flexible. In some embodiments, the carrier is weight bearing and/or flexible. In some embodiments, the carrier has the consistency of a sponge.

It will be appreciated that systems described herein will have a wide range of usefulness and applicability. In one embodiment, the carrier both contains and comprises bone grafting material. In this manner, the entire implant disposed at a desired location within the patient promotes bony growth. In a particular use, the carrier may be shaped to fit within a spinal implant used in a fusion-promoting procedure. For example, the carrier may be disposed within an opening in an interbody device placed between two adjacent vertebrae. The interbody device may comprise a metal (e.g., titanium), a plastic (e.g., PEEK), or be made from bone. In a particular use, the carrier and bone graft material is placed between adjacent spinous processes to promote fusion therebetween. Again, the carrier may be used in conjunction with a spinous process implant, such as the Aspen device commercially available from Lanx, Inc., or as a standalone device. In another embodiment, the carrier containing bone graft material is placed across the transverse processes of one or more vertebra, to promote bony ingrowth and/or fusion.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

What is claimed is:

1. A delivery system comprising:
a carrier, wherein the carrier is at least one sheet of monolithic bone tissue; and
at least one substance retained within the carrier;
wherein the carrier retains the at least one substance for implantation in a patient.

2. The delivery system of claim 1, wherein at least a portion of the carrier is perforated.

3. The delivery system of claim 2, wherein the perforated portion is formed mechanically.

4. The delivery system of claim 1, wherein the sheet of bone tissue is demineralized bone particles.

5. The delivery system of claim 4, wherein the demineralized bone particles are formed from cortical bone.

6. The delivery system of claim 4, wherein the demineralized bone particles are formed from cancellous bone.

7. The delivery system of claim 1, wherein the sheet of bone is flexible forming a flexible carrier.

8. The delivery system of claim 7, wherein the flexible carrier conforms to surrounding bony contours when implanted in a patient.

9. The delivery system of claim 1, wherein the at least one sheet of monolithic bone tissue is weight bearing to form a weight bearing carrier.

10. The delivery system of claim 9, wherein the weight bearing carrier is shaped and sized based on a surgical site for the implantation.

11. The delivery system of claim 1, wherein the carrier is configured to form at least two compartments.

12. The delivery system of claim 1, wherein the at least one substance comprises a bone grafting material.

13. The delivery system of claim 12, wherein the bone grafting material comprises osteoconductive materials.

14. The delivery system of claim 12, wherein the grafting materials are at least one of demineralized bone particles, bone fragments, bone chips, bone powder, bone matrix, mineralized allograft, growth factor, and ceramic biomaterials.

15. The delivery system of claim 1, wherein the carrier is an actively releasing material, and
wherein the at least one substance is passively released via the carrier.

16. The delivery system of claim 1, further comprising a physical attachment mechanism, wherein the physical attachment mechanism is attached to the carrier.

17. The delivery system of claim 1, wherein the carrier is a first sheet of monolithic bone tissue sealed to a second sheet of monolithic bone tissue to form at least one compartment.

18. The delivery system of claim 1, wherein the carrier is a first sheet of monolithic bone tissue folded upon itself and sealed to form at least one compartment.

19. The delivery system of claim 18, wherein the seal is selected from a group of a heat seal, an adhesion seals, a pressure seal, and sutures.

20. The delivery system of claim 1, wherein the carrier is formed in a shape selected from a following group: a ring, a cylinder, a pillow shape, a rectangular shape, a sphere, a square, and a tube.

21. The delivery system of claim 1, wherein the delivery system is adapted for promoting bone growth between two adjacent vertebrae.

22. The delivery system of claim 1, wherein the delivery system does not utilize an exogenous carrier.

23. The delivery system of claim 1, wherein the delivery system does not utilize any foreign materials promoting biocompatibility.

24. The delivery system of claim 1, wherein the delivery system minimizes migration from a site of the implantation.

25. A kit for the treatment of a bone fracture or defect, comprising:
at least one sheet of monolithic bone tissue;
at least one substance; and
a seal mechanism,
wherein the at least one sheet of monolithic bone tissue is configured to receive the at least one substance, and
wherein the seal mechanism is designed to seal the at least one sheet of monolithic bone tissue to form a compartment for retaining the at least one substance.

26. A method for treating a bone fracture or defect, comprising:
surgically placing a delivery system comprising a carrier and at least one substance retained in the carrier at the site of a bone fracture or defect;
wherein the carrier is at least one sheet of monolithic bone tissue; and
wherein the at least one substance is a bone grafting material.

27. A method for forming a delivery system, comprising:
forming a pocket in at least one sheet of monolithic bone tissue;
inserting at least one substance in the pocket;
sealing the pocket to form a carrier retaining the at least one substance.

28. The method of claim 27 wherein the forming step further comprises:
sealing a first sheet of monolithic bone tissue to a second sheet of monolithic bone tissue.

29. The method claim 27, wherein the forming step further comprises:
sealing a first portion of a first sheet of monolithic bone tissue to a second portion of the first sheet of monolithic bone tissue.

30. The method claim 27, wherein the forming step further comprises:
sealing a first portion of a first sheet of monolithic bone tissue to a second portion of the first sheet of monolithic bone tissue to form a compartment;
slicing a slit into the compartment to form the pocket.

31. The method of claim 27, wherein the forming step further comprises:
sealing a first sheet of monolithic bone tissue to a second sheet of monolithic bone tissue to form a compartment;
slicing a slit into the compartment to form the pocket.

32. The method of claim 27, wherein the sealing is performed utilizing at least one of suturing, heat, pressure, and adhesion.

33. The method of claim 27, further comprises:
perforating the at least one sheet of monolithic bone tissue before the forming step.

34. A delivery system comprising:
a carrier, the carrier including:
a demineralized cancellous bone block with a created cavity having an opening, and
an allograft derived plug, wherein the plug seals the opening of the cavity to form a compartment; and
at least one substance for promoting bone growth retained within the compartment of the carrier,
wherein the carrier retains the at least one substance for implantation in a patient.

35. A method for forming a delivery system, comprising:
forming a cavity in a demineralized cancellous bone block, wherein the cavity has an opening;
inserting at least one substance in the cavity through the opening;
sealing the opening of the cavity with an allograft derived plug to form a carrier retaining the at least one substance.

36. The method of claim 35, wherein the step of forming the cavity includes cutting the demineralized cancellous bone block with a cylindrical rotating blade to form the cavity.

37. A kit for forming a carrier, comprising:
a demineralized cancellous bone block with a created cavity having an opening; and
an allograft derived plug, wherein the plug is configured to be inserted into the opening of the cavity to seal the cavity to form a compartment.
